(19) United States
(12) Patent Application Publication
(10) Pub. No.: US 2022/0202624 A1
(43) Pub. Date: Jun. 30, 2022

(54) SELF-ADHESIVE SILICONE GEL LOADED WITH MICROPARTICLES, PARTICLES WITH IMPROVED ADHESION

(71) Applicants: Jean-Marc BRUNET, Sarcenas (FR); Christian PUSINERI, Breau-Mar (FR)

(72) Inventors: Jean-Marc BRUNET, Sarcenas (FR); Christian PUSINERI, Breau-Mar (FR)

(21) Appl. No.: 17/430,689

(22) PCT Filed: Feb. 14, 2020
(86) PCT No.: PCT/EP2020/053849

§ 371 Date: Aug. 12, 2021

(30) Foreign Application Priority Data
Feb. 14, 2019 (FR) 1901480

(51) Int. Cl. A61F 13/02; A61L 26/00; C09J 183/04; C09J 7/38

(52) U.S. Cl. CPC A61F 13/0253; A61L 26/0066; A61L 26/0019; A61L 26/0085; C09J 2301/122; C09J 183/04; C09J 7/38; C09J 2483/00; A61F 13/0266

(57) ABSTRACT

The invention relates to the use of microparticles in a self-adhesive silicone gel to increase adhesion. The invention relates to a self-adhesive silicone gel that comprises microparticles and has improved adhesion properties, in particular when same also comprises additional chemical compounds/derivatives, more particularly compounds that are active, soluble or miscible in the silicone gel.

### FIELD OF INVENTION

The present invention relates to the use of microparticles in a self-adhesive silicone gel to increase adhesion. It concerns a self-adhering silicone gel comprising microparticles and having improved adhesion properties, in particular when it also comprises additional chemical compounds/derivatives, and more particularly active compounds, soluble or miscible in the silicone gel.

### BACKGROUND OF THE INVENTION

Cosmetic or pharmaceutical compositions, in particular transdermal compositions, comprising an adhesive layer and particles are known, in particular from applications WO 2013/127929 or FR 2 781 667. These compositions may include an adhesive matrix based on silicones, which are not gels, and mineral or organic particles. However, the addition of particles to these adhesive matrices leads to a decrease in its adhesion properties to the skin, or ‘tack’. This reduction in adhesion limits the possible uses of these compositions.

The use of self-adhesive silicone gels in the medical field, for example for the production of active dressings or topical treatments is known in particular from FR 2 735 024, FR 2 879 931, WO2005/051442, WO2008/057155 and WO2017/158249. However, the introduction of liquids into adhesive silicone gels leads to a degradation of their consistency and adhesion. In addition, the degradation of their consistency and adhesion can also result from the addition of particles. US2016263270 describes adhesive silicone gels which contain powders of antibacterial agents whose content must be limited to 5% in order not to degrade the performance of the gel.

Polyaddition self-adhesive silicone gels have various applications, particularly in the medical and cosmetic fields, where they can be used as dressings or patches to be applied to the skin. These gels are well known to the profession and are the product of the hydrosilylation of two functionalized organosiloxane components in the presence of a catalyst. They are described in patents and patent applications U.S. Pat. No. 4,072,635, EP 69 451, EP 322 118, EP 737 721, FR 2 856 072, FR 2 879 931 and EP 3 356 490.

Furthermore, in certain applications silicone gel is used as a self-adhesive matrix containing an active substance, either liquid or solid, which may be in pure form or in solution in a solvent and at least part of which pure form or solution is soluble in or miscible with silicone gel. This makes it possible to impart therapeutic and/or physicochemical properties to an object made with the said gel. However, the solubilisation of a substance in a silicone gel or the incorporation of a miscible liquid into a silicone gel has the effect of greatly reducing the adhesion and consistency of the latter.

It is therefore interesting to find a solution that makes it possible to preserve or even increase the adhesion of adhesive matrices in particular for skin applications in cosmetics or for therapeutic applications, especially when the gels contain dissolved or miscible added substances.

A purpose of the invention is to make it possible to increase the adhesion of a self-adhesive silicone gel, whether or not it contains a substance at least part of which is soluble or miscible in silicone gels; the substance may be a solid, a pure or complex liquid or a solution.

### PATENT DESCRIPTION OF THE INVENTION

The present invention relates to a self-adhesive composite silicone gel (SACSG), comprising microparticles (μP) in a self-adhesive silicone gel (SASG), characterized in that it comprises by weight relative to the total weight of the SACSG at least 0.5%, in particular at least 10% of microparticles whose adsorption capacities are greater than or equal to 0.5 ml/g.

The invention relates more particularly to a SACSG which comprises at least 0.5% by weight relative to the total weight of the SACSG of porous microparticles whose adsorption capacities are greater than 1 ml/g.

The invention also concerns a pharmaceutical or cosmetic composition, characterized in that it comprises such a SACSG according to one of the claims.

It also concerns an adhesive film for cutaneous application comprising a layer (back layer), a layer adhering to the skin and a protective layer that can be removed, characterized in that the layer adhering to the skin is a SACSG according to the invention.

The invention also relates to the SACSG for its use in therapy.

It also relates to the use of particles, in particular porous particles, to increase the adhesion of self-adhering composite silicone gels, particularly on the skin.

### DETAILED DESCRIPTION OF THE INVENTION

Gels are highly deformable materials that consist of three-dimensional networks of polymers. These networks are formed by the association of polymer chains via permanent or reversible connections. There are two types of gel: chemical gels and physical gels.

Chemical gels are made up of polymer chains linked together by covalent bonds. They therefore have a high stability and the structuring of the gel is said to be irreversible.

On the contrary, physical gels are reversible. The connections are low-energy bonds, and thus depend on the thermodynamic and mechanical states of the gel.

An example of a physical gel is described in patent application WO00/73374.

In the remainder of this text, the term:
Self-adhesive gels: self-adhesive gels which have the property of adhering to a surface by light pressure.
Self-adhesive silicone gels (SASG): self-adhesive chemical gels obtained by polyaddition reaction (hyrosilylation reaction) between a polyorganosiloxane carrying alkenylsiloxy units (compound I) and a polyorganosiloxane carrying hydrogen siloxy units (compound II) in the presence of a hydrosilylation catalyst. The catalyst content makes it possible to adjust the rate of the polyaddition reaction, which can be carried out cold or hot, between 25° C. and 120° C. In this type of composition the alkenyloxy reactive groups are alkenyl groups containing 2 to 6 carbon atoms, preferably vinyl groups which are bonded to silicon; the polyorganosiloxanes carrying hydrogen siloxy units are polyorganosiloxanes carrying hydrogen atoms bonded to silicon; the catalyst is advantageously a platinum derivative.

They are conventionally constituted by the product of a hydrosilylation reaction occurring in a mixture of constituents essentially comprising:
a polyorganosiloxane I formed by a copolymer comprising units D=(R)₂SiO_{2/2} and optionally RViSiO_{2/2}, terminal units M=(R)₃Si_{1/2} and optionally M=(R)₂ViSiO_{1/2} in which the groups R, which are identical to or different from one another, are linear or branched C1-C6 alkyl and/or aryl groups, substituted or unsubstituted, and Vi representing an alkenyl radical having 2 to 6 carbon atoms and more preferably a vinyl group;
a polyhydrogensiloxane II of formula X(R)₂SiO—((R)₂SiO)ₘ—(RXSiO)ₙ—Si(R)₂X with R as defined above and X corresponding to H or R,
a platinum-based catalyst,
and optionally silicone resins.

The proportions between the alkenyl groups and the hydrogeno silane groups and the proportions between the Si—H of polydimethylsiloxanes I and II are a means of controlling the consistency and the adhesion of the gels. It is also possible to control the characteristics of formulations and gels resulting from cross-linking reactions by introducing into these formulations particular constituents such as silicone resins and by adjusting the ratios between the different constituents present in the mixtures.

They are described in particular in US patents and patent applications U.S. Pat. No. 4,072,635, EP 69 451, EP 322 118, EP 737 721, FR 2 856 072, FR 2 879 931 or EP3356490 (A1).

Preferably, the SASG according to the invention are defined in the patent application FR 2 879 931, the contents of which are incorporated here by reference. These SASG are also commercially available, in particular under the references Silpuran 2110, 2112, 2114, 2120, 2130 from Wacker, Silbione RT Gel 4317, 4642, 4717, HC2 2022, HC2 2031 from Elkem and Dow Corning MG7-9800, MG7-9850, MG7-9900 from Dow Corning, or marketed by Shinetsu or Nusil.

Silicone gels are generally characterized by a penetration value, or consistency, ranging from 90 to 350 tenths of a millimetre according to DIN ISO 2137. They are generally in the form of two-component systems which consist of a Part A and a Part B; the mixing in equidistant quantities of these two parts initiates the cross-linking reaction which leads to the formation of the silicone gel.

The viscosity and consistency characteristics of commercial silicone gels are given in Table 1 below (consistency is measured according to DIN-ISO 2137 and a cone of 62.5 g).

| | | Part A | Part B | |
|---|---|---|---|---|
| | | Viscosity | Viscosity | Consistancy |
| Reference | Supplier | mPa · s | mPa · s | mm/10 |
| Silbione RT Gel 4317 | Elkem | 3000 | 3000 | 170 |
| Silbione RT Gel 4642 | Elkem | 11000 | 15000 | 155 |
| Silbione RT Gel 4717 | Elkem | 45000 | 45000 | 170 |
| Silpuran 2112 | Wacker | 11000 | 11000 | 220 |
| Silpuran 2114 | Wacker | 12000 | 10000 | 200 |
| Dow Corning MG | Dow | 5100 | 5100 | 140 |
| 7-9900 | Corning | | | |

Non-crosslinked self-adhesive silicone gel (non-crosslinked SASG): precursor of SASG, it is a liquid composition consisting of a mixture of organosilicon derivatives and a catalyst which is capable of leading after a hydrosilylation reaction to the formation of a network of self-adhesive silicone gel (SASG). The non-crosslinked SASG are in the form of two-component systems A and B.
Self-adhesive silicone composite gel (SSCG): a self-adhesive chemical gel (SSCG), particularly an elastic material with high deformability and self-adhesive, formed by a hydrosilylation reaction, comprising, in addition to the constituents which lead to the formation of the SSCGs, the following additional constituents: porous microparticles and/or dense microparticles and, if appropriate, microspheres; active derivatives in solid and/or liquid form, solvents and/or solutions of the active derivatives and other additives. All or part of the solid active derivatives, liquid active derivatives, solvents or solutions of the active derivatives may be adsorbed by all or part of the porous microparticles.
Contents in %: the contents of the various constituents which enter into the compositions of the cross-linked or non-cross-linked SASG, or into the compositions of the cross-linked or non-cross-linked SACSG are expressed in % by weight of the overall compositions.
Microparticles (μP): particles with a size between 0.1 μm and 1000 μm which may be in the form of powders, microbeads, porous or dense, of organic, mineral or organomineral origin; these microparticles may consist of individual particles or agglomerates of smaller particles; the sizes of these microparticles may be mono-dispersed or poly-dispersed; the average sizes are between 0.1 μm and 600 μm; the diameters of the largest μPs are preferably smaller than 0.1-0.3 times the thickness of the gel layer so that the μPs are embedded in the gel layer; it is advantageous to use μPs with diameters between 3 μm and 300 μm and preferably between 5 μm and 50 μm.
Porous microparticles (μPp): Porous microparticles are well known. Pore diameters may be less than 2 nm, microporous type pores, between 2 nm and 50 nm, mesoporous type pores, or greater than 50 nm, macroporous type pores.
Impregnated microparticles (μPI)=porous μP in which all or part of the volume of the pores located on the surface or in the volume of the particles contains a liquid (solution or liquid compound or mixture of liquid compounds) and/or a solid.
Particle Adsorption Capacity (PAC): expressed in ml/g, it is measured according to the method described in standard ISO 787-5. This method consists in gradually adding linseed oil to 2-1 g of powder until a cohesive paste is obtained. A value of Adsorption Capacity is obtained which is expressed in ml linseed oil/g μP.
Total Adsorption Capacity (CAT), expressed in ml/100 g: it is calculated by the product of the quantity of particles (QμPp) in grams, in 100 g of SACSG, by the Particle Adsorption Capacity (CAP) in ml/g: CAT=QμPp×CAP ml/100 g.
The Impregnation Rate of μPI characterizes the fraction of the pore volume of the porous microparticles which is occupied by an active derivative; for a mixture of m grams of active derivative and n grams of μP, its value is given by the following expression:

(m/density of the liquid or solid)/(n×CAP)

The density is expressed in g/ml at 25° C.
The Total Impregnation Rate is defined as the ratio between the quantities expressed in ml of active ingredients and solvents present in 100 g of SACSG and the total volume of pores of the microparticles present in 100 g of SACSG; its value is given by the following relationship:

(volume(in ml) of the active derivatives[solids-liquids-solutions]present in 100 g of SACSG)/(QμPp×CAP).
Adsorption: according to the invention, it is understood that the liquids or solids penetrate into the porous particles by filling the volume of the pores of the particles; the adsorption process takes place without modification of the structure of the material which constitutes the particle.
Active agent: a pure or complex chemical derivative, liquid or solid, soluble or insoluble in the SASG-not cross-linked or cross-linked, capable of developing a biological, therapeutic or physicochemical activity.
Soluble active agent: an active derivative soluble at least 4%, between 15° C. and 120° C., in non-crosslinked and/or crosslinked SASG.
Slightly soluble or insoluble active agent: an active derivative soluble at less than 4%, between 15° C. and 120° C., in non-cross-linked and/or cross-linked SASG
Miscible solvent: a pure liquid miscible to more than 15% and up to at least 50%, between 15° C. and 120° C., in non-crosslinked and/or crosslinked SASG.
Miscible solvent: a pure liquid miscible to more than 15% and up to at least 50%, between 15° C. and 120° C., in non-crosslinked and/or crosslinked SASG.
Partially miscible solvent: a pure or complex liquid with a miscibility of between 4% and 15%, between 15° C. and 120° C., in non-crosslinked or crosslinked SASG.
Poorly miscible or immiscible solvent: a liquid whose miscibility with the non-crosslinked SASG and/or crosslinked SASG is less than or equal to 4% between 15° C. and 120° C.
Miscible liquid: a homogeneous liquid phase between 15-120° C. and at least 15% miscible with SASG-not cross-linked or cross-linked; this liquid phase consists of one or more of the preceding active agents and solvents.
Partially Miscible Liquid: homogeneous liquid phase between 15-120° C. and miscible to less than 15% and more than 4% in the non-crosslinked or crosslinked SASG; this liquid phase consists of one or more of the preceding derivatives and solvents.
Non-Miscible Liquid: liquid phase homogeneous between 15-120° C. and miscible to less than 4% with the SASG-not cross-linked or cross-linked; this liquid phase consists of one or more of the active agents and solvents.
Self-levelling: a liquid or viscous material which, when poured into an open container, forms a flat horizontal surface without the need for mechanical levelling action.
Ambient temperature: the usual temperatures 18° C. to 28° C.

The person skilled in the art will determine the content of microparticles in the SACSG according to the invention on the basis of the chemical nature of the particles, their granulometry, their adsorption capacity, the desired adhesion properties and the presence or absence of a substance or product at least partially soluble or partially miscible with silicone gel, in particular an active agent.

The microparticles and in particular the porous microparticles which may be used according to the invention are well known to the person skilled in the art; they may be of organic, mineral or organo-mineral origin, they may be hydrophobic or hydrophilic.

Among the hydrophobic porous particles, one can mention in particular the particles of organic origin obtained from:
polymethyl methacrylates such as “Sun-PMMA H”; “Sun-PMMA P”; “Sun-PMMA PH”, “Sun-PMMA X” which are manufactured by Sunjin Beauty Science or “Micropearl M 100” which are manufactured by Seppic,
polyamides such as “Orgasol 2002 U D Nat Cos” or “2002 D Nat Cos” which are manufactured by Arkema,
allyl polymethacrylate, such as “Polypore E-100”;
copolymers of allyl methacrylate and glycol dimethacrylate, such as “Polytrapp 6603”; manufactured by Health & Beauty Solutions, or
cross-linked polymethacrylate such as the “MSP-HEM-812” distributed by the Kobo company.

Particles which are hydrophilic or at least comprise surface hydroxyl groups, which comprise the hydroxyl groups present on the surface of the particles and the hydroxyl groups present on the surface of the pores, may also be used according to the invention, provided however that their possible interactions with the catalyst or another constituent of silicone gels such as polyorganosiloxanes carrying hydrogen siloxy units are taken into consideration; these reactions may furthermore be catalysed by the acidic or basic character of the particles. The selection of hydrophilic particles should therefore be carried out according to their hydroxyl group content and their acid-base character. It is also possible to use microparticles that have been “passivated”, i.e. for which the presence of hydroxyl groups has been masked beforehand and/or their surface hydroxyl content has been modified. The masking of the surface hydroxyl groups can simply be carried out when the particles are impregnated or by a substance which may preferably be poorly soluble or insoluble or still immiscible or partially miscible in silicone gels. Alternatively, particles may be used whose surface hydroxyl groups have previously been functionalized, e.g. by silylation. Such passivation methods are well known to person skilled in the art. In particular, for silica particles, a water-repellent treatment can be carried out, for example by substituting the hydrogen atoms of the groups ≡Si—OH with trimethyl silyl [—Si(CH₃)₃] groups using hexadimethylsilazane, for example. Evonik's “Aeroerl R806”, Dow Corning's “Dow Corning VM 2270”, Sunjin Beauty Science's “Sunsil 130H SC”, “Sunsil 150H SC”, Evonik's “Aerosil RX 50”, “Aerosil RX 200” are examples of treated silica particles that are commercially available. Aerosil RY 50” and “Aerosil RY 200” are examples of silica particles passivated by adsorption of silicone oils that are marketed by Evonik.

### Hydrophilic Particles Include in Particular

silica gels such as Merck's “Kieselgel”,
silica particles “Sipernat 50”, “Sipernat 60”, “Sipernat 320”, “Zeopharm 80”, “Aeroperl 300-30” from Evonik, “Sunsphere H 122” from Asahi, “Sunsil 130H”, “Sunsil 130SH”, “Sunsil 150H” from Sunjin Beauty Science,
calcium silicate particles such as Evonik's “Zeopharm 600”,
diatomaceous earth such as Celite Corporation's “Celite 266”,
calcium phosphate particles such as Fujicalin from Fuji Chemical Industry,
Calcium silicates such as “Hubersorb 250” from Huber Engineered Materials,
hydrophilic particles of organic origin such as microcrystalline cellulose particles such as the “Avicel” of the “PH” series supplied by Danisco or the Vivapur 105 and Prosolv SMCC from JRS Pharma, or cross-linked polyvinyl pyrrolidones such as the Vivapharm PVPP XL10 from JRS Pharma.

SACSG may also contain so-called dense microparticles, particles with an adsorption capacity of less than 0.75 ml/g, which may be silicone elastomer particles such as Shin Etsu's “KSP 101” and “KSP 300” or “Dow Corning 9506” and “Dow Corning 9701”; polyethylene particles such as Sumitomo's “Flo-Bead 2080” or “3040”. These dense particles can also be active particles such as Impag's “Microsilver BG” or silver salt impregnated zeolites such as Sciessent's “Agion Antimicrobial type AJ”.

The content of microparticles in the SACSG according to the invention will depend in particular on their adsorption capacity. The person skilled in the art will be able to determine the quantities of particles according to his objective of adhesion (“tack”) and/or rigidity. It has been determined that the effects of the microparticles increase as a function of the CAT. Thus, for an improvement of about 20% in adhesion and shear modulus, a CAT of 3 to 8 ml/100 g of SACSG is required.

In general, the SACSG according to the invention may comprise up to 50% of microparticles, in particular from 0.5 to 40% by weight, preferably from 0.5 to 25%. According to a particular embodiment, the SACSG comprises from 0.5 to 15% of microparticles. According to another particular embodiment, it comprises from 0.5% to 8% microparticles.

For so-called dense microparticles whose adsorption capacity is less than 0.75 ml/g, the SACSG will advantageously comprise at least 6% microparticles. For porous microparticles with an adsorption capacity of at least 0.75 ml/g, the SACSG will advantageously contain at least 0.5% porous microparticles, or even at least 10%, or even at least 2% porous microparticles, and advantageously up to 8% or even 15% or more.

When using porous microparticles, advantageously porous microparticles with an adsorption capacity (CAP) of more than 1 ml/g to more than 15 ml/g should be used. The content of these microparticles in the SACSG according to the invention will generally be from 0.5 to 30%, advantageously from 0.5 to 15%.

Porous microparticles with a CAP of at least 0.75 ml/g and up to 1.25 ml/g include “Orgasol 2002 U D Nat Cos” and “Orgasol 2002 D Nat Cos”.

According to a particular embodiment, porous microparticles have a PAC of 1.25 to 3 ml/g. In particular, mention may be made of “Celite 266”, “Kieselgel 60” HMDZ-treated, Aerosil RY 200, “Aeroperl R806” silylated silica, “Sunsphere H 122” and “Sunsphere H 122” HMDZ-treated, “Aeroperl 300-30” and “Aeroperl 300-30” HMDZ-treated, “Sun PMMA-P” and “Sun PMMA PH”.

According to another embodiment, the porous microparticles have a PAC of at least 3 ml/g. In particular, mention may be made of “Polypore E100”, “Sipernat 50” and “Sipernat 50” treated with HMDZ, “Sun PMMA-X”, “Polytrap 603” and “Dow Corning VM 2270”.

In general, porous microparticles have an average particle size of 0.1 μm to 600 μm, preferably less than 500 μm, more preferably less than 100 μm. The skilled person in the art will determine the appropriate particle size according to the thickness of the materials to be prepared from the SACSG and the viscosity of the non-crosslinked SACSG; in order to limit the increase in viscosity of non-crosslinked SACSG, it is preferable to use μP with larger particle sizes. Advantageously, microparticles have an average particle size of at least 0.1 μm, in particular from 0.1 μm to 300 μm, more particularly from 1 μm to 100 μm, advantageously from 5 μm to 50 μm.

According to a preferred embodiment, porous microparticles have an average particle size of between 5 μm and 50 μm.

According to an embodiment, mixtures of microparticles with different average particle sizes and/or different CAP may be used.

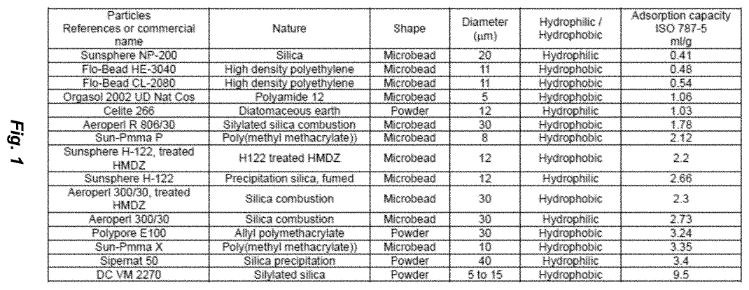

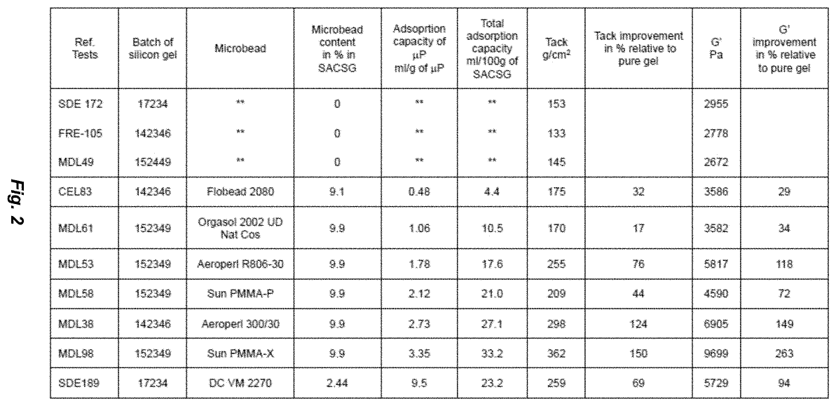

Table 2 in FIG. 1 gives a non-exhaustive list of commercially available microparticles and their properties.

One way for the skilled person to determine the quantity of particles used in the SACSG according to the invention is to calculate the Total Adsorption Capacity (TAC) of all the particles used per 100 g of SASG.

Advantageously, the SACSG according to the invention has a CAT greater than or equal to 3 ml per 100 g of SACSG. According to a particular embodiment, the SACSG has a CAT of at least 5 ml/100 mg, which may be as high as 30 ml/100 mg, or even higher.

The invention also relates to a SACSG as previously defined which also includes one or more additional chemical compounds/derivatives, and more particularly active compounds, soluble or miscible in silicone gel. These chemical compounds, in particular active agents, can be advantageously adsorbed in the pores of the microparticles. They can be liquid at room temperature, miscible or partially miscible with SASG or with one of its constituents. When the liquid chemical compound is little or not miscible with the cross-linked or non-cross-linked SACSG, it can be solubilized in a solvent miscible with the cross-linked or non-cross-linked SACSG to form a solution which will be at least partially miscible with the cross-linked or non-cross-linked SACSG. They may also be solid and at least partially soluble in the cross-linked or non-cross-linked SASG. When the solid additional chemical compounds/derivatives are sparingly or not at all soluble in the cross-linked or non-cross-linked SASG they may be dissolved in a solvent miscible with the cross-linked or non-cross-linked SASG; this solution shall form a liquid at least partially miscible with the cross-linked or non-cross-linked SASG.

These chemical compounds, in particular these active agents may be used in cosmetics or therapy, depending on the biological or therapeutic effects sought.

The active agents used in cosmetics are well known to the skilled person, notably marketed in the products of companies specialising in the manufacture and marketing of cosmetic products such as L'OREAL, PROCTER & GAMBLE, UNILEVER, SHISHEIDO, CLARINS, CAUDALIE, tec.

Cosmetic active agents may be chosen for their antiseptic, antibiotic, emollient, moisturizing, photo-protective, astringent, exfoliating, skin and cell renewal stimulating, slimming, skin absorption promoting activity.

They can be of natural or synthetic origin.

They can be in the form of plant powder or nebulisates of plant extracts, concrete oils, oleoresins, essential oils, oily extracts, liposoluble molecules. These ingredients must be compatible with the SACSG according to the invention, and will be chosen in particular among the following derivatives:
extracts in particular in powder form and nebulisates such as those of aloe, turmeric, citrus, Centella asiatica, mimosa tenuiflora,
Essential oils in general and in particular, essential oils of thyme, wintergreen, peppermint, turmeric, helicrysum.
vegetable or animal oils rich in saturated fatty acids, for example palm oil, coconut oil rich in palmitic acid, myristic, stearic acid;
vegetable or animal oils rich in monounsaturated fatty acids: e.g. palmitoleic or oleic acids, in particular hazelnut, olive and/or mustard oils
vegetable or animal oils rich in polyunsaturated fatty acids, in particular those rich in linoleic acid (omega3) and linolenic acid (omega6), docosahexaenoic acid, eicosapentaenoic acid, docosahexaenoic acid, arachidonic acid, such as sunflower, grape seed, sesame, macadamia, cumin, canola, fish oil, argan, safflower, argemone, passionflower, calophyllum, chaulmoogra oils, etc,
oils of animal origin such as shark or mink oil richin squalane, squalene, essential fatty acids, vitamins A, D, E, liquid or acetylated lanolin,
oily extracts: chamomile oil, St. John's wort, arnica, rose hip oil, evening primrose oil, calendula oil rich in liposoluble active ingredients such as retinol, alpha tocopherols and its derivatives, phytosterols, arnicin, hypericin, faradiol,
synthetic oils, in particular linear or branched esters: purcelin, esters of myristic acid, isopropyl palmitate or lanilate, decyl isostearate, butyl stearate, propylene glycol dipelargonate, decyl oleate, oleate, octyldodecanol isopropyl palmitate, myristyl stearate, octyl palmitate, butyl arichidonate isostearyl isostearate, isostearyl neopentanoate, miglyol, acetylated lanolin; or polyol esters: palmitate, stearate, propylene or diethylene glycol isostearate, glycol caprylate, polyethylene glycol stearate or isostearate,
oleoresins from capsicum, turmeric, pepper,
lipoamino acids such as dipalmitoylhydroxyproline, palmitoylkeratin, undecylenoylglycine, caprilylcholine,
liquid surfactants such as oxyethylenated fatty alcohols: laureth 4, sorbitan diisostearate,
antibiotics: erythromycin, clindamycin palmitate,
preservatives: parabens, especially butyl parabens, isothiazolinone, dibromodicyanobutane,
antioxidants: alpha tocopherol and its acetate, ascorbyl palmitate, rosemary and ginkgo extracts, in particular ginkgolic acid, butylhydroxy toluene, butylhydroxyanisole, propyl gallate, octyl gallate,
absorption promoters: propylene glycol esters such as propylene glycol dilaurate, 4 cyclohexyl benzyl methyl nicotinate, dimethyl sulfoxide, xylene, isopropyl myristate, azone, glycol palmitate, polyol esters such as octanoates or decanoates of glycerol or ethylene glycols, esters of trimellitic acid such as tridecyl trimellilate, esters of palmitic acid such as erythritol tetra palmitate, esters of stearic or isostearic acid such as glycol tri stearate or glycol tri isostearate,
exfoliants: azelaic acid, ivy or burdock extracts, kojic acid, agaric acid, ursolic acid, adapalene, retinoids,
antiseptics: chlocresol, benzyl benzoate; fatty acid triglycerides with 4 to 18 carbon atoms.

The active agents used for their therapeutic activities are well known to the skilled person, especially those used by cutaneous or transdermal administration. Examples of active agents according to the invention include antibacterial agents, antiseptics, antivirals, keratolytics, antioedematous agents, antinaupathic agents, protein and thyrosine inhibitors, kinase, anticholinergics, monoclonal antibodies, withdrawal agents, anti-anxiety agents, local anesthetics, analgesics, antimycotic agents, antiacne agents, sedatives and tranquilizers, anxiolytics, hormones, androgenic steroids, estrogenic steroids, progestin steroids, analgesics, hypoglycemics, antispasmodics, beta-blockers, non-steroidal anti-inflammatory drugs, steroidal anti-inflammatory drugs, anti-osteoporotic agents, skin whitening agents, vasodilators, antihypertensives, antiparkinsonian drugs, migraine drugs, antineoplastics, vitamins, essential amino acids, essential fatty acids.

They may be in powder or liquid form, or in adsorbed, absorbed or encapsulated form. These ingredients will preferably be compatible with the SACSG according to the invention, and in particular:
Bactericides: beta-diketone metal chelates such as pentane dione chelates, 3-Octylpentane-2,4-dione chelates of silver, copper and cerium, cineol 1-8, thymol, linear or branched fatty acid salts of silver, copper, cerium such as stearate, isostearate, palmitate, oleate of silver, copper, cerium, salts and oxides of Zinc, Copper, Cerium, Titanium.
Antiseptics: alkyl benzoate, chlorocresol, chloroxylenol,
antibiotics: mupirocin, fusidic acid, clindamycin palmitate, tiamulin, erythromycin,
steroidal anti-inflammatory drugs: betamethasone, triamcinolone, dexamethasone,
non-steroidal anti-inflammatory drugs: ibuprofen, indomethacin, methyl salicylate, diclofenac, enoxolone,
antioedematous agents: menthol, helienaline,
antinaupathics: metopimazine, domperidone, scopolamine,
protein kinase inhibitors such as everolimus; thyrosine kinase inhibitors such as temsirolimus, afatinib; monoclonal antibody inhibitors such as trastuzumab,
anticholinergics: rivastigmine,
withdrawal agents: nicotine, methadone, buprenorphine,
vasodilators: hederogenin methyl ester, verapamil, propanolol, candesartan, clonidine,
the anti-anxiety drugs: trinitrine, isosorbide laurate,
antivirals: adefovir, dipevoxil,
local anaesthetics: butamben, prilocaine,
Painkillers: fentanyl, tramadol, capsaicin,
antimycotics: econazole, terbinafine, clotrimazol,
anti-acne medication: benzoyl peroxide,
sedatives, tranquilizers and anxiolytics: mirtazapine, hydroxyzine, prazepam, venlafaxine, dimenhydrate, acetophenazine, haloperidol,
hormones and antihormones: cyproterone, thyroxine, triamcinolone, aldosterone, tamoxifen, equol,
steroid hormones: androgens such as testosterone or androsterone, oestrogens such as oestriol or ethynylestradiol, progestins such as progesterone,
antispasmodics: trimebutine, trifluoroglucinol, atropine, benzatropine, papaverine,
diuretics: furosemide,
antiparkinsonian drugs: adamantine, arachidonoyl dopamine,
migraine medications: pizotifen, dihydroergotamine,
bleach: hydroquinone benzyl ether,
antineoplastics—imiquimod, fluouracil, bexarotene,
immunosuppressants: tacrolimus,

It is understood that the active ingredients identified for each category above are identified as representative examples of the class of activity, but are in no way intended to limit the list. The person skilled in the art shall know, as necessary, how to identify and use other active ingredients of a given category to implement the invention.

The active agents may be used alone or in mixtures.

The SACSG according to the invention may include:
at least 50% silicone gel,
at least 2.5% by weight, preferably at least 5%, of one or more additional compounds in solubilized form or of one or more additional compounds in liquid form or of a solution of one or more additional compounds,
at least 0.5% by weight of microparticles with an Adsorption Capacity greater than or equal to 1 ml/g.

The amount of additional chemical compounds/derivatives will be determined according to the use of the prepared SACSG, and the nature of the additional compound in combination with the microparticles to be selected.

It has been observed that the addition of these soluble or miscible compounds will decrease the adhesion and stiffness properties of the SACSG. The person skilled in the art will therefore be able to determine the quantities of microparticles and soluble or miscible compounds according to his objective, both in terms of the content of additional compounds and the adhesion and rigidity properties sought according to the use that will be made of the SACSG.

Thus, the SACSG according to the invention makes it possible to obtain silicone gels which comprise from 5% to 40% by weight of liquid compounds or in solutions which have adhesion and rigidity properties at least equal to those of the corresponding pure silicone gel, i.e. without microparticles and without additional compounds.

According to a particular embodiment the SACSG may contain up to 10% to 25% by weight of additional chemical compounds/derivatives, in particular active agents, relative to the total weight of SACSG, from 15 to 20% by weight.

These percentages apply both to the additional compounds alone or to the solutions containing them. For an additional compound alone, in particular active agent alone or a mixture of active agents alone, the percentage applies to the compound alone or to the mixture of compounds. If the additional compound must be solubilized in a compatible solvent to be miscible with SACSG, then the percentage applies to the solution containing the additional compound(s), in particular the active agent(s).

These additional chemical compounds, in particular of active agents alone, in mixtures or in solution, may be introduced directly into one of the constituents of the SASG or by the introduction of microparticles which will have been previously impregnated with these active agents or solutions of active agents. When the active agents are poorly/not very soluble or poorly/not miscible in/with the SASG, they should preferably be introduced in the form of solutions in a solvent so as to be able to form a liquid which will be at least partially miscible with the SASG.

Solvents which are miscible with the constituents of the SACSG according to the invention and more particularly usual solvents suitable for cosmetic or pharmaceutical use, shall be used as solvents. These solvents may, if necessary, themselves be active ingredients due to their properties. Particular mention should be made of organic solvents such as isopropyl myristate, isopropyl palmitate, isotridecyl isononylnonanoate, neopentyl glycol dioctanoate, branched parafins such as isododecane; cyclic silicone derivatives which consist of 4, 5, 6, 7 siloxyl units; phenyl derivatives of silicones such as diphenylsiloxy phenyl trimethycone; organofunctional silicones. One can also mention, for example, comb copolymer type copolyols which comprise a polydimethylsiloxane chain on which are grafted polyether chains or KF-6038 which consists of a polydimethyl siloxane chain to which polyether chains and alkyl chains are grafted and is marketed by Shinetsu. Liquid actives which are at least partially miscible or miscible with cross-linked or non-cross-linked SASG can also be used as solvents.

The SACSG according to the invention may also contain, together with the additional chemical compounds, liquid, mixed or in solution, other additional compounds such as dense microparticles of solid actives, microcapsules, etc.

In this case, the SACSG according to the invention will advantageously comprise from 5 to 40% of additional compound(s).

According to a particular mode, the SACSG will comprise at least 50% of silicone gel, at least 5% of an additional chemical compound, at least 10% of microparticles whose adsorption capacity is greater than or equal to 1 ml/g, in particular from 1 to 15%, and from 5 to 30% of additional compound(s).

The person skilled in the art will be able to determine the total quantity of liquid (solution, liquid additional compound or mixture of liquid additional compounds) in the SACSG according to the invention as a function of the desired adhesion properties, but also due to the microparticles and the chemical compatibility, miscibility, of the liquid with the silicone gels.

In particular, it is possible to identify a maximum ratio between the contents of liquid or solid active derivatives and the contents of μP identified by a Total Impregnation Rate (TIR) determined according to the following formula:

[amounts (in ml) of liquids or solids]/[amounts of μP(in g)×μP adsorption capacities(in ml/g)].

The quantities correspond to those present in 100 g of SACSG.

When SACSG do not contain dense microparticles of solid active ingredients, the TIT value must be advantageously lower than a value between 0.45 and 1.2.

When the SACSG according to the invention are used to manufacture films or patches, they advantageously have a thickness of 0.015 mm to 5 mm, preferably 0.015 to 3 mm. This thickness may be of several cm when SACSG are used to manufacture moulded parts.

SACSG according to the invention can be prepared by one of the following methods:
direct mode 1: first parts A and B are mixed, then the desired quantity of microparticles is introduced into the mixture (A+B); the mixture [(A+B)+microparticles] is homogenized, the desired quantity of liquid phase is added to the mixture [(A+B)+microparticles], the mixture (A+B)+microparticles]+liquid phase is homogenized and then degassed under 20-50 mBar for 10 minutes; the degassed mixture is used to obtain the desired adherent material. It is also possible to add the liquids before the microparticles. When the composition does not contain microparticles, the liquid phase is added to the mixture (A+B), this last mixture is homogenized and then degassed.
Indirect mode 1: according to this process, the microparticles are added successively to each of Parts A and B, then the miscible or partially miscible liquid phase is added, the mixtures are homogenized after each addition and then degassed at 20-50 mbar; Parts A′ and B′ are obtained, which will contain microparticles and the miscible or partially miscible liquid phase. As far as possible, equivalent amounts of microparticles and active agents and liquids shall be added to each of Parts A and B. These Parts A′ and B′ may then be mixed, after degassing at 20-50 mbar these mixtures are used to prepare the SACSG. This method can also be used to introduce a partially soluble solid by adding the same quantities of this solid to each of Parts A and B, thus obtaining Parts A′ and B′; in the latter case it may be advantageous to subject Parts A′ and B′ to heat treatments in order to facilitate the solubilisation of a solid and/or the distribution of the constituents.

When the substance to be introduced into the mixture [(A+B)+microparticles] is a solid, preferably direct mode 2 or indirect mode 2 is used; these modes can also be advantageously used to prepare SACSG from liquid-impregnated microparticles.
direct mode 2: in this case microparticles which are used have been previously impregnated with active agents, solid or liquid, and/or solutions of active agents.

These impregnated particles are used as in the case of direct mode 1. Parts A and B are first mixed, then the desired quantity of impregnated microparticles is added to the mixture (A+B); the mixture (A+B)+impregnated microparticles is homogenized, the desired quantity of any additional liquid phase is added to the mixture [(A+B)+impregnated microparticles], the mixture [(A+B)+impregnated microparticles]+possibly liquid phase is homogenized and then degassed at 20-50 mbar for 10 minutes; the degassed mixture is used to obtain the desired adherent material.
Indirect mode 2: this time microparticles which have been previously impregnated with the active agents or solutions of active agents are introduced into each ofparts A and B, it is also possible to add other constituents in the same way. The mixtures are homogenized and degassed under the conditions just described in the preceding cases. After degassing, parts A′ and B′ are obtained which are used to prepare SACSG. In the case of this indirect mode 2, it may be advantageous to subject mixtures A′ and B′ to heat treatments.

These modes of preparation are given for information only. The person skilled in the art will be able to adapt them according to particular constraints, in particular the presence or absence of volatile derivatives, the distribution of impregnated or non-impregnated μP, active agents, liquids or solvents in a heterogeneous manner in parts A and B, etc.

The mixtures [A+B+microparticles], [A+B+microparticles+active and/or liquid agents] as well as the mixtures [A′+B′] can be implemented by casting, injection and/or coating techniques in order to be able to lead, after cross-linking, to materials which can be handled: films, coated fabrics, moulded parts, etc. In the context of the targeted applications, formulations are preferred where the viscosities of the mixtures [A+B+microparticles+active and/or liquid agents] or the mixtures [A′+B] are between 1000 and 500,000 mPa·s or at least correspond to a self-levelling mixture at a temperature of between 15° C. and 120° C. In the case where the mixtures are used according to a coating technique on a support, compositions with a viscosity between 25 000 mPa·s and 50 000 mPa·s are preferred.

This viscosity will depend on the combination of parts A and B, the CAT and the content of active and/or liquid agents.

In the case where the formulation contains liquid ingredients of volatile character, the cross-linking reaction must be able to be carried out under conditions of temperature and time compatible with the thermal stability and volatility of the liquid ingredients; silicone formulations are preferred whose cross-linking can be carried out at temperatures below 120° C. and preferably between 40° C. and 80° C.; the reactivity of the mixtures can be adjusted by varying the platinum catalyst contents.

The SACSG according to the invention can be used in multiple fields due to their advantageous properties, both in cosmetics and in therapy or in any industry which has a use for such SACSG. Particular mention should be made of the therapeutic uses, particularly transdermal, in cosmetics, for protective parts for orthoses and prostheses, for damping, electrical insulation, assembly, etc.

The SACSG according to the invention can be prepared in various forms according to their applications, occlusive strips, liquid-permeable strips, films, moulded parts, composite parts (reinforced SACSG parts), etc.

The invention also relates to an adhesive or patch dressing for skin application comprising an outer layer (back layer) which is arranged on the side of the SACSG opposite to that which will be applied to the skin, a layer adhering to the skin, characterized in that the layer adhering to the skin is a SACSG according to the invention as defined above.

For the storage of the dressing, it also comprises a peelable protective layer on the side of the SACSG intended to be applied to the skin.

The outer layers are made of the materials customary in the field. They are chosen according to their mechanical properties, their compatibility with silicone gels, their permeability to water vapour and their ability to act as a barrier to UV rays; they must also be impermeable to active ingredients. These materials may be selected from thermoplastic polymers such as high or low density polyethylenes, polypropylenes, copolymers of ethylene and vinyl acetate, polyethylene terephthalates, or complexes of these materials with aluminium foil. The carrier may also be an elastic material such as polyurethanes or silicone elastomers. Such elastic supports may also be made of composite materials formed by combinations of an elastic fabric and a polyurethane sheet as described in EN 2904214 or application EP0976383.

SACSG according to the invention can also be used to make liquid-permeable dressings such as those described in U.S. Pat. No. 4,921,704.

SACSG according to the invention may also be used for the manufacture of moulded parts, composite parts (reinforced gels) or coated textiles, in particular for the manufacture of prostheses, orthoses, etc. which are also part of the present invention.

### EXAMPLES

### Materials and Methods

1. Preparation of Materials for Performance Characterization.
Characterization of rheological properties: for these characterizations, a ˜0.6 g sample of non-crosslinked SACSG should be placed in the measuring cell. In the case of mixtures obtained by the direct mode, a sample of the degassed mixture whose preparation has just been described is used. If Parts A′ and B′ which have been obtained by the indirect method are available, both Parts A′ and B′ are rehomogenised if necessary, equivalent masses of A′ and B′ are weighed and mixed with a spatula; after degassing at 20-50 mbar at room temperature until the bubbles are eliminated (about 10 minutes), a sample of the mixture may be taken and placed in the measuring cell. When parts A′ and B′ do not contain the same quantities of additional compounds, the weighing of A′ and B′ must take these differences in composition into account.
Adhesion characterization (Tack): in this case 2 mm films of the cross-linked SACSG must be prepared. Depending on their densities, 3.5 to 5.5 g of the degassed mixtures, whose preparation has just been described, are poured into a transparent polystyrene Petri dish with a diameter of 5.6 cm. If necessary, a second vacuum degassing is carried out. After replacing the lid of the dish, the dish is then placed on a flat and horizontal support for 24 hours at a temperature of 20-26° C. and then for 48 hours at 70° C. to complete the cross-linking. After cross-linking, a self-adhesive film is obtained whose surface is flat and parallel to the bottom of the Petri dish; the thickness of this film is equal to 2±0.2 mm. When the actives or solvents can degrade or solubilize the polystyrene, the polystyrene Petri dishes are replaced by glass Petri dishes with flat bottoms with diameters of 5.3 cm.

In addition, when the formulations contain volatile constituents, the dishes are sealed by applying a Teflon tape; then after the first phase of cross-linking at room temperature on the horizontal plate, the dishes are introduced into a container which can be closed tightly before being treated at 70° for 48 hours. This procedure minimises losses during the two phases of cross-linking; the contents of volatile derivatives can be checked by subjecting the films to drying at 700 in an oven.

2. Characterization of Gel Adhesion

Since patches are one of the main applications for which these self-adhering silicone layers are intended and since in this type of application the patches are applied to the skin without exerting strong pressure, the Tack measurement was preferred as a means of characterizing adhesion. Tack is classically characterized according to the so-called “Probe Tack” test described in ASTM D 2979. It consists of applying to the surface of the silicone gel film a cylindrical rod 5.2 mm in diameter and then measuring the peel force when the rod is vertically torn from the surface of the gel film. The rod is applied to the surface of the gel film until a force of 2N is reached (application speed 10 mm/min), after holding the rod for 1 s, the rod is vertically torn off from the surface at a speed of 250 mm/min. The Tack corresponds to the maximum value of the tear-off force that is recorded. This test procedure was developed for the characterization of thin films of adhesives with thicknesses well below 100 μm. In the case of thicker layers (thickness from 500 μm to more than 2 mm) this method is no longer applicable.

The measurement of tack is therefore carried out using the Probe Tack method, whose test procedure is derived from that described above in the case of ASTM 2979.

The device used is the Rheometer RS-6000 marketed by the Thermofischer company equipped with a 10 mm diameter rod. The roughness of the rod end meets the criteria of the ASTM 2979 standard.

The samples are discs of 2 mm thickness and 54 mm or 50 mm diameter which are respectively prepared extemporaneously in polystyrene Petri dishes of 56 mm diameter or in glass Petri dishes of 53 mm diameter according to the method described above. The 2 mm thick samples contained in the Petri dishes are attached to the Rheometer stage.

Test Procedure:

Zero determination: An empty Petri dish is attached to the rheometer plate. The 10 mm diameter rod is lowered vertically at a speed of 50 mm/min. until contact is made with the bottom surface of the Petri dish (detection by the instrument of a force other than zero); the position of the rod is recorded by the instrument and corresponds to the 0 dimension of the rod.

Tack measurement: the Petri dish is replaced by a box containing the 2 mm thick disc whose Tack must be determined. The 10 mm bar is lowered vertically at a speed of 3 mm/min until a force of 0.2N is applied to the surface of the sample. This force of 0.2N is maintained for a period of 30 seconds and then the rod is pulled vertically out at a speed of 150 mm/min.

The Tack is expressed by the maximum value of the tear-off force and is expressed in g/cm².

Three measurements are made on three different samples, and under these conditions standard deviations of less than or equal to 10 are obtained.

3. Measurement of Gel Consistency

The consistency of the gels is characterized by their elastic shear modulus, which is measured as follows:
rheometer: Thermofisher RS 6000.
Plan-Plan cell: rotor with 20 mm radius, rotor/stator gap=0.5 mm.
mode: imposed stress=10 Pa.
oscillation frequency: 1 Hz.
temperature: sample installation at 30°.
closing of the cell in two steps: 3 mm/mn until D=1.5 mm, then 0.15 mm/mn until D=0.5 mm.
temperature rise to 70° in 60 s.
measurement G′-G″ at 70° for 4000 s, return to 30°.

This type of measurement makes it possible to characterize the value of the elastic component of the shear modulus G′, as well as the gel time which corresponds to the time which elapses between the moment when the temperature of the sample reaches the value of 70° C. and the moment when the value of G′ becomes higher than that of G″ (G″ corresponds to the loss component of the shear modulus). When the gels develop high damping capacities which result in a value of the ratio G″/G′ equal to 1 after total cross-linking, the value of G′ at 4000 s is assimilated to that of G″ at 4000 s. These values are expressed in Pa.

4. Measurement of the Adsorption Capacity

Adsorption capacities are measured according to the method described in ISO 787-5. This method consists of gradually adding linseed oil to 2-1 g of powder until a cohesive paste is obtained. A value of Adsorption Capacity is obtained which is expressed in ml linseed oil/g μP.

### EXAMPLES OF APPLICATIONS

### 1. Effects of Adsorption Capacities

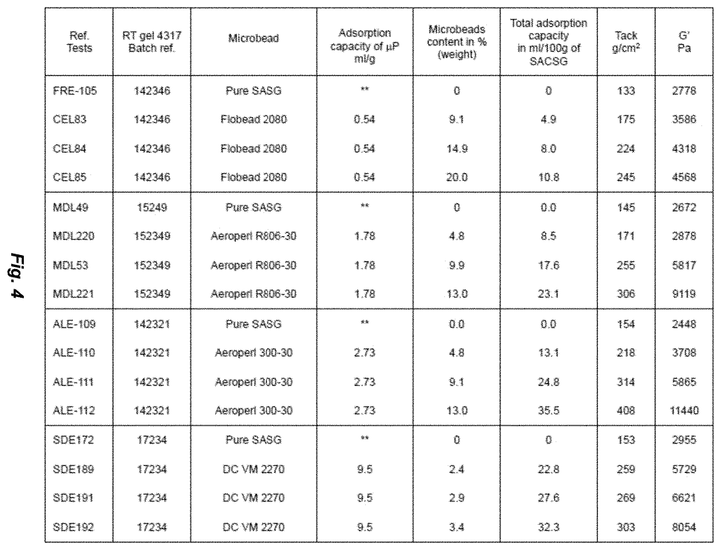

The following examples describe the effects of the addition of 2.4% to 9.9% μP in SACSG compositions, which are described in Table 4 in FIG. 3 (different μP and μPp which are described in Table 2 in FIG. 1), on the performance of SASG bearing the references RT Gel 4317 and Silpuran 2112. SACSG containing 2.4% to 9.9% microparticles were prepared using direct mode 1.

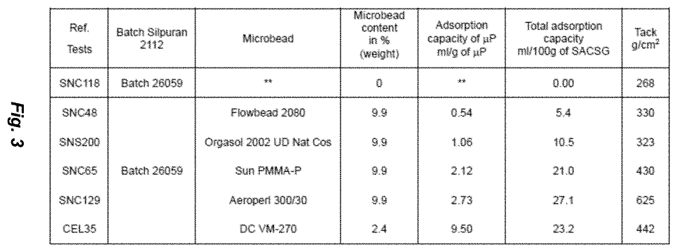

Table 3 in FIG. 2 describes the compositions, characteristics and performance of SACSG-based on RT Gel 4317. These results show that the addition of μP increases the Tack or Elastic Shear Modulus values by more than 50% and that the higher the Adsorption Capacity of the microparticles, the greater the improvement in adhesion and consistency.

Table 4 in FIG. 3 relates to the SACSG that have been obtained from Silpuran 2112. The results in Table 4 show that the addition of microparticles to this gel leads to the same type of results: the addition of microparticles leads to an improvement in adhesion, the effect of the microparticles increases with their Adsorption Capacity, it is even possible to multiply the adhesion of Silpuran 2112 by a factor of more than 2.

### 2 Effects of Microparticle Contents and CAT

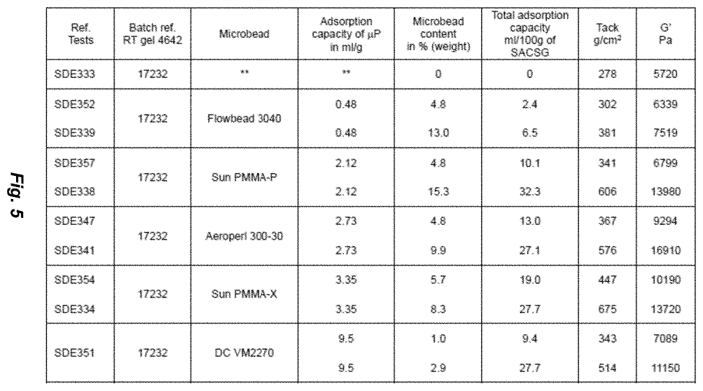

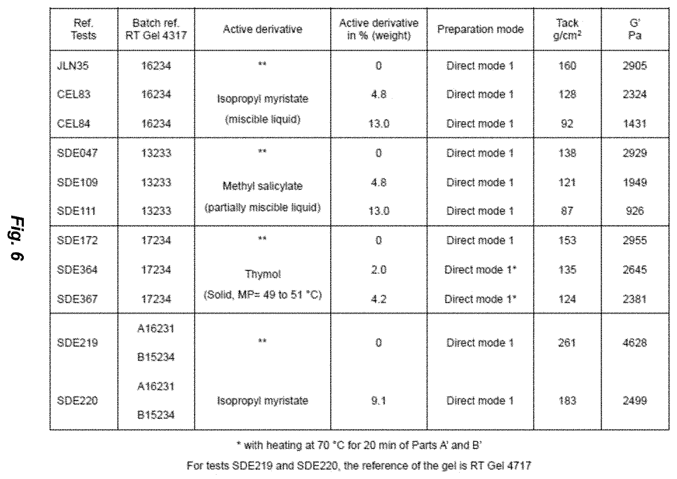

The following examples describe the influence of adding different amounts of particles to the following two SASG: RT Gel 4317 and RT Gel 4642. The ASG were prepared using direct mode 1; Tables 5 and 6 in FIGS. 4 and 5 describe the results obtained with RT Gel 4317 and RT Gel 4642, respectively.

All these results show that the effects of the microparticles increase systematically with their contents and adsorption capacities. From the data concerning the compositions, it is possible to calculate the Total Adsorption Capacity (TAC) which is equal to the content of microparticles in the SACSG multiplied by their Adsorption Capacities. These results also show that globally the values of Tack and G′ increase with the values of the CAT.

### 3. Degradation of Performance by Introduction of a Liquid or Solubilization of a Solid

The following examples describe the degradation of SASG performance by the introduction of miscible or partially miscible liquids or solubilization of a solid. In these tests Isopropyl Myristate, Methyl Salicylate and Thymol are examples of a fully miscible liquid, a partially miscible liquid and a partially soluble solid in SASG, respectively. Isopropyl Myritate is more than 50% miscible with SASG, the miscibility of Methyl Salicylate is limited to 12-15%, the solubility of Thymol is between 4 and 5%.

Samples which contain Isopropyl Myristate and methyl salicylate have been prepared in the direct-1 mode using glass Petri dishes; in the case of methyl salicylate, this derivative is volatile, samples which are intended for Tack measurements must be prepared under the special conditions described above. In the case of Thymol, a variant of the indirect-1 mode has been used; this derivative is a solid with a melting point of 55° C.; in order to facilitate its solubilization in silicone gel, the following procedure is used: to each of the Parts A and B the appropriate quantities of Thymol are added; thus Parts A′ and B′ are obtained which contain the same quantities of Thymol; these Parts A′ and B′ are vialed; the stoppered flasks are placed for 20 minutes in a heated enclosure to 70°; each of the Parts A′ and B′ is then homogenized and kept in the stoppered flasks; after return to room temperature it is observed that Parts A′ and B′ are transparent and homogeneous. Parts A′ and B′ are used as described in the procedure relating to active derivatives of volatile character which has just been described in the case of methyl salicylate.

Thymol is crystalline Thymol supplied by Roth, Lot 14110049/B; Methyl Salicylate is the pure derivative supplied by Cooper, Lot 15050195/E; Isopropyl Myristate is supplied by Roth, Lot 306199473. These are also the same products that were used in all of the examples in Tables 3 to 15.

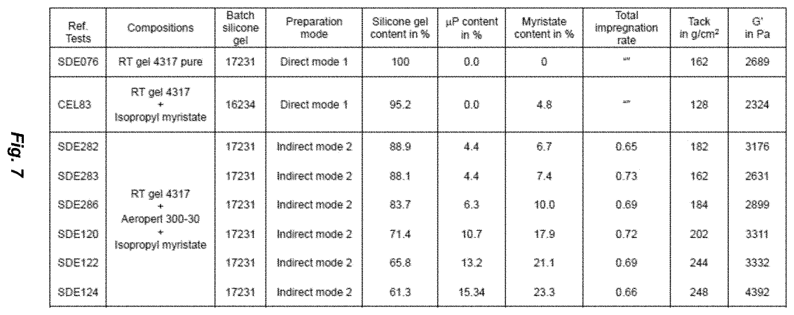

The results presented in Table 7 in FIG. 6 show that the introduction of the active derivatives systematically decreases the performance of SACSG compared to controls (pure gels).

### 4. Effect of Microparticles According to the Invention

### 4.1 RT Gel 4317/Aeroperl 300-30/Myristate Compositions

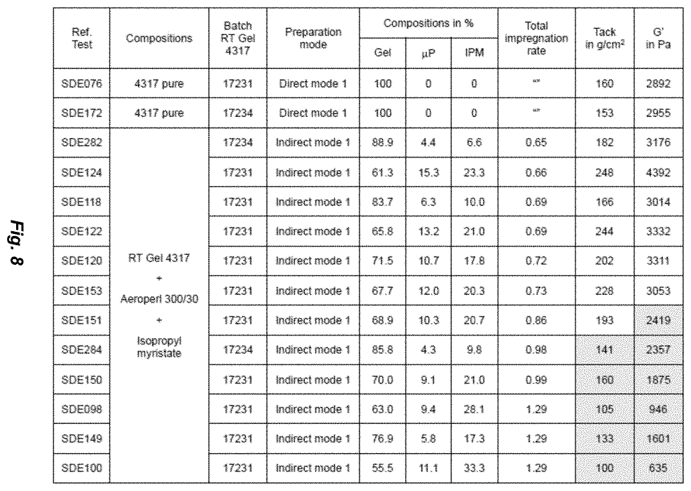

Table 8 in FIG. 7 describes the results for mixtures of “RT Gel 4317/Aeroperl 300-30/Isopropyle Myristate”. These samples were prepared in indirect mode-1 as follows: to each of Parts A and B the microparticles are added successively (dried for 24 hours at 70°), after dispersion of the μPs, isopropyl myristate is added, the mixture is homogenised; thus Parts A′ and B′ are obtained which are degassed under vacuum and then bottle; the stoppered flasks are then placed for 24 hours in a chamber thermostatically controlled at 70° C. Parts A′ and B′ are then used to prepare the samples which are intended for Tack and shear modulus characterizations.

The results shown in Table-8 show that the introduction of microparticles compensates for the effects of the presence of isopropyl myristate; while the presence of 4.8% Isopropyl Myristate causes the values of Tack and G′ to drop by 20% (see Table 7), the presence of Aeroperl 300-30 makes it possible to obtain SACSG containing from 6% to 23% Isopropyl Myristate whose values of Tack and G′ are greater than or equal to those of pure gel.

However, for a given amount of microparticles the amount of Myristate must not exceed certain limits beyond which the microparticles can no longer compensate for the effects of isopropyl Myristate additions as shown in the examples in Table 9 in Figure-8. These examples relate to compositions containing 55-89% RT Gel 4317, 4-13% Aeroperl 300-30 and 6-33% Isopropyl Myristate. The SACSG were prepared using the specific indirect mode 1 which is described for the tests shown in Table-8.

These results indicate that for this type of composition, the Total Impregnation Rate value must be less than 0.98-0.86 in order for the Tack and G′ values to be equal to or greater than those of the pure gel.

### 4.2 RT Gel 4317/Dow Corning VM 2270/Myristate Compositions

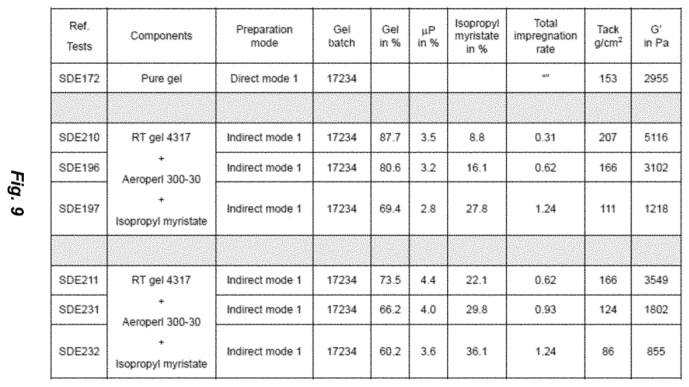

The same type of result is obtained with the Dow Corning VM 2270 microparticles reported in Table 10 in FIG. 9.

Tests were conducted under conditions identical to those described for RT Gel 4317/Aeroperl 300-30/Myristate mixtures. The results are similar: it is possible to prepare compositions containing 23% Isopropyl Myristate whose performance will be at least equal to that of pure gel and the saturation rate value must remain below a limit value.

### 4.3 Other Examples

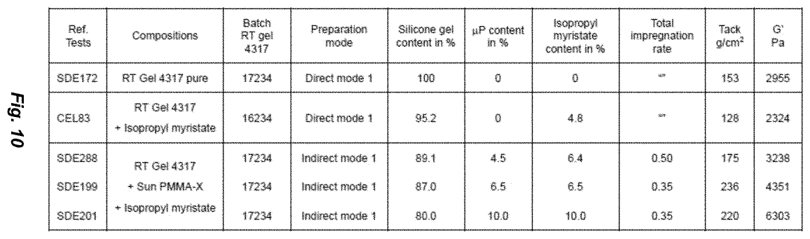

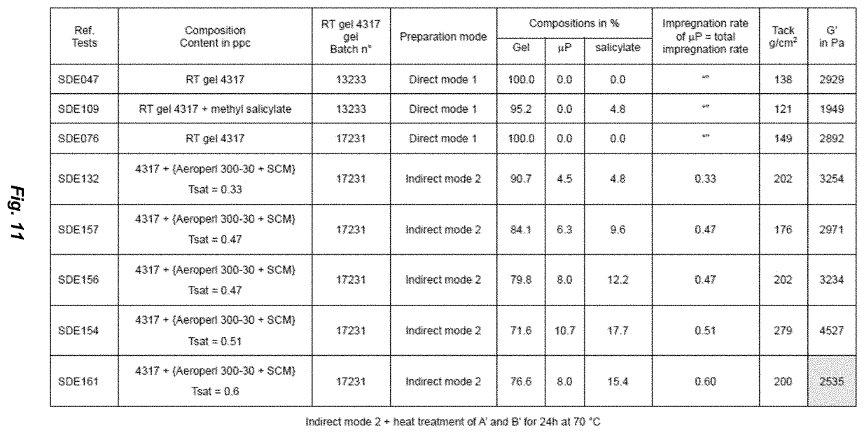

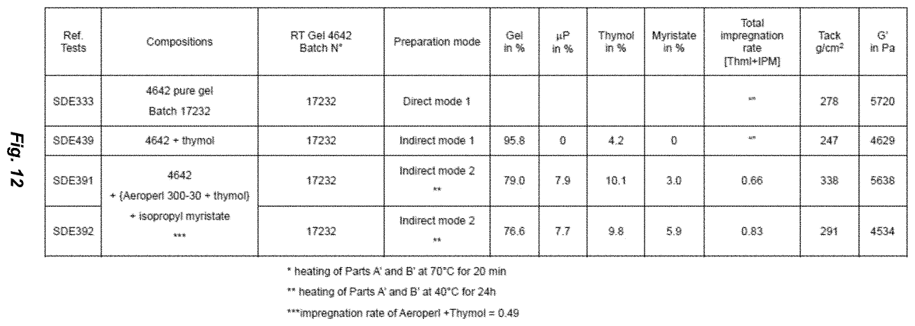

Tables 11 to 13 respectively in FIGS. 10 to 12 give similar results for other examples of gels, particles and chemical compounds.

Table 11 “RT Gel 4317/Sun PMMA-X/Isopropyl Myristate” The products for the characterization of Tack and G′ were prepared using indirect mode 1, but subjecting parts A′ and B′ to a 48-hour heat treatment at 70° to facilitate the distribution of isopropyl myristate.

The results show that all mixtures containing between 6-10% isopropyl myristate and 4.5-10% of the Sun PMMA-X microparticles have Tack and G′ values which are higher than those of the pure gel.

Table 12. RT Gel 4317/{Aeroperl 300-30/Methyl Salicylate}

These mixtures have been prepared in indirect mode 2 using Aeroperl 300-30 impregnated with methyl salicylate; the impregnation rates of Aeroperl 300-30 are 0.33, 0.47, 0.51 and 0.6. In the case of these mixtures the Impregnation Rates of the microparticles are equal to the Total Impregnation Rates. These tests have been carried out according to indirect mode 2, with the addition of a 24-hour heat treatment at 70° C. of Parts A′ and B′. In the case of the tests which were carried out with μP whose Impregnation Rate is less than 0.6, compositions are obtained which contain from 4.8 to 17.7% methyl salicylate and whose performance is equal to or better than that of pure gel. In the case of test SDE-161 which was obtained with pp whose Impregnation Rate is 0.6 only the value of Tack is higher than that of the gel; the value of the elastic component of the shear modulus, G′, is just 12% lower than that of pure gel.

Table 13: “RT Gel 4642/{Aeroperl 300-30+Thymol}/Isopropyl myristate: Table 13 shows the results that were obtained from compositions that contain:
a/RT Gel 4642, microparticles impregnated with Thymol (at an impregnation rate of 0.49) and isopropyl myristate.
b/a mixture of RT Gel 4642 and Thymol.

RT Gel 4642/{Aeroperl 300-30+Thymol}/Isopropyl myristate mixtures are prepared according to indirect mode 2 by adding to each of Parts A and B the same quantities of impregnated microparticles and then isopropyl myristate. Parts A′ and B′ are subjected to a heat treatment at 40° for 24 hours in stoppered vials. The 2 mm films are prepared under the conditions described in the case where the SACSG contain volatile derivatives.

The mixture RT Gel 4642 and Thymol is prepared according to indirect mode 1 by adding the same quantities of crystalline Thymol to each of Parts A and B; Parts A′and B′ are then heated at 70° C. for 20 min in stoppered vials. The 2 mm films are also prepared under the conditions which are described in the case where the SACSG contain volatile derivatives.

These heat treatments melt the Thymol or promote the distribution of the constituents of the mixtures.

The results shown in Table 13 lead to findings similar to those made previously. The addition of 4.2% Thymol to RT Gel 4642 degrades the performance of the gel (test SDE 439). As shown in SDE 391, the presence of 7.9% microparticles makes it possible to obtain SACSG that contain a mixture of 13% Thymol and Isopropyl Myristate while developing performances that are at least equal to those of the pure gel; in the case of this test, the value of the Total Impregnation Rate is 0.66. In the case of test SDE-392 where the Total Impregnation Rate value is 0.83 the values of tack and G′ are lower than those of the pure gel; this result also shows that the Total Impregnation Rate value must be below a limit value.

1. A self-adhering composite silicone gel, comprising microparticles in a self-adhering silicone gel, wherein the said self-adhering composite silicone gel comprises by weight relative to the total weight of the self-adhering composite silicone gel at least 0.5% of microparticles whose adsorption capacities are greater than or equal to 0.5 ml/g.

2. The self-adhering composite silicone gel according to claim 1, comprising at least 0.5% by weight relative to the total weight of the self-adhering composite silicone gel of porous microparticles whose adsorption capacities are greater than or equal to 1 ml/g.

3. The self-adhering composite silicone gel according to claim 1, comprising up to 50% of microparticles.

4. The self-adhering composite silicone gel according to claim 1, comprising up to 30% of microparticles.

5. The self-adhering composite silicone gel according to claim 1, wherein the porous microparticles have an adsorption capacity of 2 to 15 ml/g.

6. The self-adhering composite silicone gel according to claim 1, wherein the porous microparticles have an average particle size of 0.1 μm to 600 μm.

7. The self-adhering composite silicone gel according to claim 1, comprising additional chemical compounds.

8. The self-adhering composite silicone gel according to claim 7, wherein the additional chemical compounds are active agents.

9. The self-adhering composite silicone gel according to claim 7, wherein the additional chemical compounds are miscible or soluble in the self-adhering composite silicone gel or one of its constituents.

10. The self-adhering composite silicone gel according to claim 9, wherein the additional chemical compounds are miscible in a content of at least 4% with the self-adhering silicone composite gel or one of its constituents, or soluble in a content of at least 4% with the self-adhering silicone composite gel or one of its constituents.

11. The self-adhering composite silicone gel according to claim 7, wherein the additional chemical compounds are in solution in a solvent miscible with the self-adhering silicone composite gel.

12. The self-adhering composite silicone gel according to claim 7, having the following composition:
at least 50% by weight of silicone gel,
at least 2.5% by weight of one or more additional compounds in solubilized form or of one or more additional compounds in liquid form or of a solution of one or more additional compounds, and
at least 0.5% by weight of microparticles with an Adsorption Capacity greater than or equal to 1 ml/g.

13. The self-adhering composite silicone gel according to claim 1, having a thickness of 0.015 mm to 5 mm.

14. An adhesive dressing for skin application comprising:
an outer layer (back layer),
a layer adhering to the skin, and
a peelable protective layer,
wherein the layer adhering to the skin is a self-adhesive composite silicone gel according to claim 1.

15.-16. (canceled)

17. The self-adhering composite silicone gel according to claim 1, wherein the porous microparticles have an average particle size of less than 100 μm.

18. The self-adhering composite silicone gel according to claim 7, having the following composition:
at least 50% by weight of silicone gel,
at least 5%, of one or more additional compounds in solubilized form or of one or more additional compounds in liquid form or of a solution of one or more additional compounds, and
at least 0.5% by weight of microparticles with an Adsorption Capacity greater than or equal to 1 ml/g.

19. The self-adhering composite silicone gel according to claim 1, having a thickness of 0.015 mm to 3 mm.
